(19) United States
(12) Patent Application Publication
(10) Pub. No.: US 2006/0257434 A1
(43) Pub. Date: Nov. 16, 2006

(54) Production of emulsions of pharmaceutical compositions

(76) Inventors: Mark Mugerditchian, Poway, CA (US); Maria S. Bruno Figueroa, Guaynabo, PR (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/407,512

(22) Filed: Apr. 20, 2006

Related U.S. Application Data
(60) Provisional application No. 60/674,080, filed on Apr. 22, 2005.

(51) Int. Cl. A61K 9/00

(52) U.S. Cl. 424/400

(57) ABSTRACT

Disclosed are methods of producing an emulsion comprising determining a desired final pH of the emulsion, mixing an oil, surfactant, stabilizer, and a water-insoluble pharmaceutical, adjusting the pH of the mixture, and homogenizing the mixture, such that the starting pH of the mixture, the rotation speed of the homogenizer, and the temperature at which the homogenization is carried out are adjusted to give the desired pH.

### RELATED APPLICATIONS

The present application claims priority to U.S. Provisional Application Ser. No. 60/674,080, filed on Apr. 22, 2005, by Mugerditchian et al. and entitled “PRODUCTION OF EMULSIONS OF PHARMACEUTICAL COMPOSITIONS,” which is hereby expressly incorporated by reference in its entirety.

### FIELD OF THE INVENTION

The present invention relates to methods of producing an emulsion with a final pH suitable for use in intravenous delivery of a water-insoluble pharmaceutical.

### SUMMARY OF THE INVENTION

Disclosed is a method of producing an emulsion comprising determining a desired final pH of the emulsion, mixing an oil, surfactant, stabilizer, and a water-insoluble pharmaceutical, homogenizing the mixture to create an emulsion, adjusting the rotation speed of the homogenizer, the temperature at which the homogenization is carried out, and the pH of the emulsion to give the desired pH. Disclosed is a method of producing an emulsion comprising determining a desired final pH of the emulsion, mixing an oil, surfactant, stabilizer, and a water-insoluble pharmaceutical, homogenizing the mixture to create an emulsion, adjusting the rotation speed of the homogenizer, the temperature at which the homogenization is carried out, and the pH of the mixture, to give the desired pH.

### DETAILED DESCRIPTION OF THE PREFERRED EMBODIMENT

Different classes of compounds are known to have diuretic effects, and are thus useful in the treatment of patients suffering from fluid overload. Diuretics act on specific segments of nephrons, the functional units of the kidney. Some xanthine derived compounds, such as caffeine, constitute one class of diuretics. The diuretic properties of these xanthine derivatives are due to their ability to interfere with the action of adenosine. Adenosine produces a vasoconstrictive effect in afferent arterioles in the kidney, resulting in a decrease in renal blood flow and glomerular filtration rate. Adenosine also has a role in the phenomenon known as tubuloglomerular feedback, which occurs when an acute increase in sodium levels in the proximal tubule of the nephrons feeds back to decrease glomerular filtration. Adenosine works via both adenosine A₁ and A₂ receptors. Certain xanthine derivatives are a subclass of adenosine A₁ Receptor Antagonists, (“AA₁RA's”), and possess potent diuretic and renal protective activities. AA₁RA's decrease afferent arteriolar pressure, and increase urine flow and sodium excretion. While AA₁RA's possess valuable diuretic properties, certain AA₁RA's are notoriously insoluble in water. KW-3902 is an example of an AA₁RA. Over a physiological pH range, the solubility of KW-3902 is less than 1 μg/ml. Hosokawa, T. et al., Chem. Pharm. Bull. 50(1) 87-91 (2002), herein incorporated by reference in its entirety. As used herein, the term “water insoluble” refers to compounds that have solubility of less than or equal to about 1 μg/ml in water.

Often, it is desirable to deliver AA₁RA's intravenously. Due to their low solubility, the manufacture of pharmaceutical compositions of AA₁RA's that are both suitable for intravenous injection and that minimize adverse side effects in patients has proven particularly challenging. Traditional approaches to deliver water-insoluble compounds intravenously included solubilizing the drugs using detergents or organic solvents, creating a solution of the water-insoluble drug by adjusting the pH outside the physiological range, or utilizing molecular complexes with a vehicle. However, several of these approaches presented undesirable side-effects in patients, such as local pain or precipitation of drugs after injection.

The use of dispersed systems such as emulsions, (e.g., oil-in water emulsions), provides an alternative approach to overcome the problems encountered with traditional approaches to delivery of water-insoluble drugs. Emulsions are mixtures of two normally immiscibile liquids, in which one exists as tiny particles within the other. Oil-in-water emulsions consist of colloidal suspensions of oil droplets, in which the water insoluble compound is dissolved and homogenously dispersed through the water. The oil droplets are reduced in size to such a degree that the oil's normal repulsion of the water molecule is overcome by the minute size of the droplets.

Emulsion systems by their nature are thermodynamically unstable. Thus, stabilizers are used to enhance the formation and stability of oil-in-water emulsions. Amphipathic molecules, which have polar and non-polar moieties, are useful in stabilizing the particles in the emulsion such that the particles do not coalesce. Changes in the stability of the emulsion can manifest in various ways, such as changes in particle size of oil droplets and changes in bulk pH. Surfactants are examples of stabilizers. The term “surfactant” as used herein refers to substances which change the nature of a surface, including water surface tension. Surfactants are often classified as anionic, cationic, non-ionic hydrophilic (polar), non-ionic lypophilic (non-polar), or amphoteric (possessing acidic and basic properties). Amphipathic surfactants have the ability to interact with both the water and oil components of the emulsion, and their ability to function as stabilizers can be attributed in part to this characteristic.

In preparation for creating an oil-in-water emulsion, in some embodiments of the present invention, a water insoluble pharmaceutical is mixed with an oil. In some embodiments, the oil is a triglyceride. Triglycerides, or triacylglycerols, are composed of glycerol and fatty acid chains, having the structure CH₂COOR—CHCOOR′—CH₂—COOR″, wherein R, R′, and R″ are fatty acids. Fatty acids are chains of carbon atoms connected by single bonds alone (saturated fatty acids) or by both single and double and/or triple bonds (unsaturated fatty acids). In some embodiments, the oil is a monoglyceride while in other embodiments, the oil is a diglyceride.

The acid component of the fatty acid is more water soluble than the hydrocarbon chain. Thus, the shorter the hydrocarbon chains are in a fatty acid, the more water soluble the fatty acid is.

With regard to emulsions used for parenteral delivery of drugs, particular attention is given to the particle size of the emulsion. Large oil droplets could give rise to blockages in the body, and thus smaller particle size is desirable. The particle size of emulsions of pharmacologically active compounds also affects the clearance of the emulsion from the blood. In general, fine particle size emulsions are cleared more slowly than coarse particle size emulsions. Davis, S. et al., “Medical and Pharmaceutical Applications of Emulsions”, in Encyclopedia of Emulsion Technology, Vol. 2, Paul Becher, Ed., © 1995, Marcel Dekker, Inc., New York, N.Y., pp. 159-235, herein incorporated by reference in its entirety. In oil-in-water emulsions of pharmacologically active compounds, bioavailability of the active compound is affected by the surface/volume ratio of the emulsion. Particle size thus affects the bioavailabilty, since the surface/volume ratio is inversely related to the particle size.

While oil-in-water emulsions are an attractive alternative for intravenous delivery of water insoluble drugs, several parameters affect their stability. Changes in stability will affect drug release and drug release may in turn affect stability. Davis, S. et al., supra. Oil-in-water emulsions can be sensitive to pH, particle size, and temperature. Aspects of the present invention provide a predictable method of producing an emulsion suitable for the delivery of pharmaceuticals, having a desired particle size and pH, obviating the need to adjust the pH in the final emulsion.

Aspects of the present invention are directed to methods of producing an emulsion for intravenous injection of a water-insoluble pharmaceutical composition by mixing an oil, a surfactant, and a stabilizer, with the water-insoluble pharmaceutical composition to obtain a mixture, homogenizing the mixture in a high shear homogenizer in a bath at a certain temperature to create an emulsion, and adjusting the pH of the emulsion, such that the parameters of the target pH, rotation speed of the homogenizer, and bath temperature are adjusted to obtain a final pH of the emulsion between 5 and 7. “Target pH” refers to the pH of the mixture immediately after adding either an acid or base. “Final pH” refers to the pH of the emulsion prior to use such as preparing an ampule or such as injection into a patient. In some of the embodiments described herein, the target pH is adjusted to yield a predetermined final pH.

In another aspect, methods of producing an emulsion for intravenous injection of a water-insoluble pharmaceutical composition are disclosed wherein an oil, a first surfactant, a stabilizer, and a water-insoluble pharmaceutical are mixed to obtain a mixture. The pH of the mixture is adjusted to a target pH, and the mixture is homogenized in a high shear homogenizer in a bath at a certain temperature, such that the parameters of the target pH, rotation speed of the homogenizer, and bath temperature are adjusted to obtain a final pH of the emulsion between 5 and 7. In some embodiments, the pH of the mixture is adjusted to a target pH during the homogenization step. In still other embodiments, acid or base can be added more than once to adjust the target pH. For example, acid or base can be added prior to and during the homogenization step.

The steps in the above methods can be practiced in an order other than the order given. For example, acid or base can be added to adjust the pH to a target pH following the mixing step, and prior to the homogenization step. In some embodiments, the pH is adjusted to a target pH following the homogenization step and prior to the microfluidization step. In other embodiments, the pH is adjusted to a target pH following mixture of the oil, first surfactant, stabilizer, and water-insolube pharmaceutical, and prior to homogenization. In yet other embodiments, the pH is adjusted to a target pH during the homogenization step.

In embodiments of the invention where the oil is a triglyceride, the triglyceride is naturally occurring or optionally synthetic. In some embodiments, the triglyceride comprises at least one fatty acid chain that is greater than or equal to 8 carbons in length. In other embodiments, the triglyceride comprises at least one fatty acid chain this is less than 22 carbons in length. Thus, in certain embodiments, the fatty acid chains of the triglyceride are about 8-22 carbons in length. Examples of naturally occurring triglycerides include, but are not limited to vegetable oils, such as soybean oil, safflower oil, olive oil, and cottonseed oil. In embodiments of the invention where the oil is a monoglyceride, the monoglyceride is naturally occurring or optionally synthetic. In some embodiments, the synthetic monoglyceride comprises a fatty acid chain that is about 8-22 carbons in length. In embodiments of the invention where the oil is a diglyceride, the diglyceride is naturally occurring or optionally synthetic. In some embodiments, the diglyceride comprises at least one fatty acid chain that is greater than or equal to 8 carbons in length. In other embodiments, the diglyceride comprises at least one fatty acid chain this is less than 22 carbons in length. Thus, in certain embodiments, the fatty acid chains of the diglyceride are about 8-22 carbons in length.

Embodiments of the present invention encompass the different classes of surfactants, including but not limited to, amphoteric surfactants. In some embodiments the surfactant contains phosphorous. Examples of phosphorous containing surfactants include, but are not limited to naturally occurring phospholipids and PEG-phospholipids. Regarding pharmaceutical compositions, the use of naturally occurring surfactant molecules may be desirable, in that it may reduce the risk of undesirable biological reactions in the patient. Naturally occurring phospholipids include, but are not limited to egg yolk lecithin, which is known to consist of phosphatidylcholine, phosphatidylinositol, and phosphatidylethanolamine. Other embodiments of the invention include the use of purified phosphatidylcholine. Use of phosphorous containing surfactants now known or later discovered is within the scope of the present invention.

In other embodiments, the surfactant comprises block copolymers. For example, some embodiments of the invention include, but are not limited to, polyoxyethylene-polyoxypropylene (PLURONICS®). With respect to the present invention, acceptable surfactants are nontoxic to recipients, such as patients, at the dosages and concentrations employed.

In some embodiments of the invention, the stabilizer comprises a surfactant, including but not limited to non-ionic surfactants. Examples of non-ionic surfactants include, but are not limited to, sorbitan esters of fatty acids (such as SPAN®), polyethylene glycol (“PEG”) ester (such as BRIJ®), PEG fatty acid esters (such as CREMOPHOR®), PEG-sorbitan fatty acid esters (such as TWEEN®), and fatty alcohols, and cholesterol. The term “ester” as used herein refers to compounds possessing an (R′—COOR″) functional group. The structure of esters is such that they can function as hydrogen-bond acceptors, but cannot act as hydrogen-bond donors. Consequently, esters are more water soluble than cognate hydrocarbons and more hydrophobic than cognate alcohols or acids.

Polyethylene glycol is a polymer of ethylene oxide, having the structure:
—(CH₂—CH₂—O)_{N}—

PEG is soluble in water and is often coupled to hydrophobic molecules to produce non-ionic surfactants. PEG-based surfactants are useful in pharmaceutical compositions as they are non-toxic.

In other embodiments of the invention, chelating agents, antioxidants, salt-forming counter-ions, and buffers are used as stabilizers. In other embodiments, the stabilizer is an oncotic agent.

The term “oncotic agent” refers to a compound that functions to control oncotic pressure, which arises due to the presence of colloids on one side of a semi-permeable barrier. Oncotic agents function to equalize the pressure inside and outside the permeable barrier, e.g., a cell membrane, so to minimize changes in water balance across the semi-permeable barrier. Oncotic agents are desirable when limiting the use of ions, such as salts, to adjust or maintain the pressure across a semi-permeable membrane is desirable. Examples of oncotic agents include, but are not limited to, hydrophilic compounds, glycerin, saccharides, sugar alcohols, and polypeptides.

In some embodiments of the invention, the water insoluble pharmaceutical composition is an adenosine A₁ receptor antagonist (AA₁RA). Examples of A₁ receptor antagonists include, but are not limited to xanthine derivatives. KW-3902 is a xanthine-derived A₁ receptor antagonist. The chemical name of KW-3902 is 8-(3-noradamantyl)-1,3-dipropylxanthine, also known as 3,7-dihydro-1,3-dipropyl-8-(3-tricyclo[3.3.1.0^{3,7}]nonyl)-1H-purine-2,6-dione, and its structure is 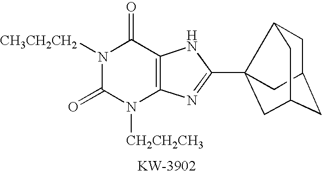
Thus, in one embodiment of the present invention, the water-insoluble pharmaceutical composition is KW-3902. Other AA₁RA's suitable for use with the methods described herein include those listed in International Publication No. WO 2004/075856 and International Publication No. WO 2004/096228, both of which are herein incorporated by reference in their entirety.

Embodiments of the present invention include compositions having emulsifiers. In some embodiments the emulsifier is an organic acid. The organic acid may have more than five carbon atoms, more than 10 carbon atoms, or more than 15 carbon atoms. In some embodiments, the organic acid has at least one double bond. In one embodiment, the organic acid is oleic acid. In other embodiments the emulsifier is a monoglyceride, including acetylated monoglycerides, or a diglyceride. Other embodiments of the present invention include non-ionic surfactants, including but not limited to the examples listed above, such as a PEG-sorbitan fatty acid ester/sorbitan fatty acid ester mixture (TWEEN®/SPAN®) as an emulsifier.

In some embodiments the pH of the mixture of compounds above is adjusted to a target pH, by the addition of an acid or base. In some embodiments of the present invention, the target pH is at least 6.0. In other embodiments of the invention, the target pH is at least 6.3. In yet other embodiments of the invention, the target pH is at least 7.0, 7.3, 7.5, 8.0, 8.5, or 9.0.

Mechanical shearing of a mixture, for example in a homogenizer, is one method to create an emulsion. Following the adjustment of the pH of the mixture comprising the compounds above, the mixture can be homogenized to produce a crude emulsion. In some embodiments of the invention, the rotation speed of the homogenizer can be between 5,000 and 18,000 rotations per minute (rpm). In other embodiments of the invention, the rotation speed can be between 6,000 and 9,000 rpm's. In yet other embodiments the rotation speed can be between 7,000 and 8,000 rpm's. In some embodiments, the pH can be adjusted to a target pH by the addition of acid or base following homogenization. When the target pH is reached, the mixture can be homogenized again. In some embodiments, the second homogenization step yields the final emulsion.

Some embodiments of the present invention relate to performing the homogenization of the crude emulsion at a controlled temperature by performing the homogenization in a bath. In some embodiments of the present invention, the temperature of the bath is at least 25° C. In other embodiments of the invention, the bath temperature is at least 30° C. In yet other embodiments, the bath temperature is at least 35° C. In yet other embodiments of the invention, the temperature of the bath is at least 40° C. In yet other embodiments of the invention, the temperature of the bath is no more than 45° C.

Droplet size of emulsions is a parameter that relates in part to stability of the emulsion. In cases where the water insoluble compound exists primarily at the interface of the oil/water surface, smaller particle size results in higher chemical potential of the compound. In some embodiments of the invention, the average particle size of the crude emulsion following homogenization is at least 100 nm. In other embodiments of the invention, the average particle size of the crude emulsion is at least 150 nm. In yet other embodiments, the average particle size of the crude emulsion is at least 200 nm, at least 250 nm, at least 300 nm, at least 350 nm, at least 400 nm, or at least 450 nm.

In some instances, it may be desirable to reduce the average particle size of the crude emulsion, or reduce the distribution of the mean particle size of the crude emulsion following homogenization. Thus, another aspect of the present invention relates to reduction of the average particle size of the crude emulsion, to obtain a final average particle size by passing the crude emulsion through a microfluidizer. In some embodiments, microfluidization is required. In some embodiments of the present invention the crude emulsion is passed through a microfluidizer at least five times. In other embodiments of the invention, the crude emulsion is passed through a microfluidizer at least three times. In yet another embodiment of the invention, the crude emulsion is passed through a microfluidizer at least two times.

### EXAMPLES

### Example 1

### Effect of Temperature and Starting pH on Final pH of KW-3902 Emulsion

The following reagents were mixed together (Table 1):

TABLE 1

| | | | | mg per vial | Amount |
|---|---|---|---|---|---|
| Component | Reference | Function | mg/mL | (20 mL) | per Batch |
| KW-3902 | In-House | Active Ingredient | 0.5 | 10 | 50 g |
| | Standard (DSM | | | | |
| | Pharmaceutical | | | | |
| | Chemicals) | | | | |
| Refined Egg Yolk | In-House | Emulsifier | 50 | 1000 | 5 kg |
| Phosphatidylcholine | Standard (NC-50) | | | | |
| (R-EPC) | | | | | |
| Soybean Oil | USP (CRODA, | Solvent | 50 | 1000 | 5 kg |
| | Inc) | | | | |
| Oleic Acid | JPE (NOF Corp) | Emulsifier | 2.4 | 48 | 240 g |
| Concentrated Glycerin | JP (NOF Corp) | Oncotic Agent | 22.1 | 442 | 2.21 kg |
| Water for Injection | USP | Vehicle | q.s.* to | q.s. to | q.s. to |
| | | | 1 mL | 20 mL | target total |
| | | | | | volume |
| Nitrogen | NF | Head-space gas | q.s. | q.s. | q.s. |
| | | | | Total | 100 L |

*q.s.: quantity sufficient

The mixture was homogenized at either 7,000 or 8,000 rpms, using a Silverson Machine high shear homogenizer model L4RT, for 30 minutes. To assess the effect of temperature on particle size and pH of the final emulsion, the homogenization step was performed at 26° C., 32° C., or 40° C. Following the homogenization step, Sodium hydroxide and Hydrochloric acid were added to adjust the pH of the mixture, to 6.3, 7.3, or 8.3 (“target pH”), as measured by an Accumet, Model 50 pH Meter from FisherScientific. The crude emulsions were passed through a Model M-110EH microfluidizer (Microfluidics Corp., Newton, Mass., USA) three times at 120 MPa. The final pH of the fine emulsions were measured, and the data are shown in Table 2.

TABLE 2

| Lot | Temperature (° C.) | Target pH | Final pH |
|---|---|---|---|
| 2556-02-31A | 26.0 | 7.3 | 6.0 |
| 2556-02-31B | 40.0 | 8.3 | 6.9 |
| 2556-02-31C | 33.0 | 7.3 | 6.7 |
| 2556-02-31D | 33.0 | 8.3 | 7.1 |
| 2556-02-31E | 40.0 | 6.3 | 5.9 |
| 2556-02-31F | 26.0 | 8.3 | 6.7 |
| 2556-02-31G | 33.0 | 6.3 | 6.0 |
| 2556-02-31H | 40.0 | 7.3 | 6.5 |
| 2556-02-31I | 26.0 | 6.3 | 6.4 |

As the target pH was increased, the final pH of the emulsion increased. The effect of the bath temperature on the final pH depended on the target pH. At a target pH of 6.3, the final pH decreased as the bath temperature increased. To the contrary, at a target pH of 8.3, the final pH increased as the bath temperature increased. At a target pH of 8.3, the final pH is about 7.0. The mixture does not need to be cooled, as bath temperature had little effect on final pH in this range.

### Example 2

### Effect of Rotation Speed on Particle Size and pH of KW-3902 Emulsion

The ingredients listed in Table 1 were mixed together, and were homogenized in a Silverson Machine high shear homogenizer model L4RT, for 30 minutes at either 7,000 or 8,000 revolutions per minute (rpm's). Next, the target pH was adjusted to 8.3 with sodium hydroxide and hydrochloric acid. The emulsion was then passed through a Microfluidics microfluidizer model M-110EH either three or five times at 120 MPa. The final pH was measured. Mean particle size was measured using a 90Plus Particle Sizer, from Brookhaven Instrument Corp. The data are shown in Table 3.

TABLE 3

| | Speed | Processing | | Mean Particle Size |
|---|---|---|---|---|
| Lot | (rpm's) | cycle | Final pH | (nm) |
| 2556-02-31J | 8000 | 3 | 6.4 | 153.3 |
| 2556-02-31K | 7000 | 3 | 6.1 | 174.7 |
| 2556-02-31L | 8000 | 5 | 6.3 | 154.4 |
| 2556-02-31M | 7000 | 5 | 6.6 | 163.8 |

The rotation speed of the high shear mixer appears to affect the particle size. As the rotation speed was increased, the particle size decreased. Rotation speed and mean particle size were not accurate predictors of final pH.

### Example 3

### Effect of Target pH and Temperature on Particle Size

The ingredients listed in Table 1 were mixed together, and were homogenized in a Silverson Machine high shear homogenizer model L4RT, for 30 minutes at either 7,000 or 8,000 revolutions per minute (rpm's). Next, the target pH was adjusted to 8.3 with sodium hydroxide and hydrochloric acid. The emulsion was then passed through a Microfluidics microfluidizer model M-110EH either three or five times at 120 MPa. The final pH was measured. Mean particle size was measured using a 90Plus Particle Sizer, from Brookhaven Instrument Corp. The data are shown in Table 4.

| | | | Mean Particle Size |
|---|---|---|---|
| Lot | Temperature (° C.) | Target pH | (nm) |
| 2556-02-31A | 26.0 | 7.3 | 113.9 |
| 2556-02-31B | 40.0 | 8.3 | 115.6 |
| 2556-02-31C | 33.0 | 7.3 | 120.5 |
| 2556-02-31D | 33.0 | 8.3 | 119.6 |
| 2556-02-31E | 40.0 | 6.3 | 105.5 |
| 2556-02-31F | 26.0 | 8.3 | 117.9 |
| 2556-02-31G | 33.0 | 6.3 | 152.2 |
| 2556-02-31H | 40.0 | 7.3 | 149.5 |
| 2556-02-31I | 26.0 | 6.3 | 149.1 |

### Example 4

### Effect of Number of Passes Through a Microfluidizer on Particle Size and Particle Distribution of KW-3902 Emulsion

The ingredients listed in Table 1 were mixed together, and were homogenized in a Silverson Machine high shear homogenizer model L4RT, for 30 minutes at either 8,000 revolutions per minute (rpm's). Next, the target pH was adjusted to 8.3 with sodium hydroxide and hydrochloric acid. The emulsion was then passed through a Microfluidics microfluidizer model M-110EH as indicated at 120 MPa. The final pH was measured. Mean particle size was measured using a 90Plus Particle Sizer, from Brookhaven Instrument Corp. The data are shown in Tables 5-18.

TABLE 5
(Average Particle Size)

| Lot | Premix | 1 Pass | 2 Passes | 3 Passes | 4 Passes | 5 Passes | Speed (rpm's) |
|---|---|---|---|---|---|---|---|
| 2556-02-31A | 259.1 | 132.8 | 126.8 | 126.2 | 130.0 | 113.9 | 8000 |
| 2556-02-31B | 171.6 | 128.7 | 131.8 | 120.2 | 115.0 | 115.6 | 8000 |
| 2556-02-31C | 197.0 | 136.2 | 127.8 | 126.8 | 121.3 | 120.5 | 8000 |
| 2556-02-31D | 196.0 | 137.0 | 131.2 | 131.2 | 148.7 | 119.6 | 8000 |
| 2556-02-31E | 255.0 | 123.1 | 115.5 | 115.2 | 107.3 | 105.5 | 8000 |
| 2556-02-31F | 263.2 | 139.1 | 125.1 | 124.0 | 109.2 | 117.9 | 8000 |
| 2556-02-31G | 442.2 | 170.2 | 172.8 | 165.8 | 157.0 | 152.2 | 8000 |
| 2556-02-31H | 203.5 | 158.2 | 152.6 | 153.1 | 151.4 | 149.5 | 8000 |
| 2556-02-31I | 349.4 | 177.4 | 161.0 | 159.0 | 154.8 | 149.1 | 8000 |
| 2556-02-31J | 393.2 | 181.6 | 169.2 | 153.3 | | | 8000 |
| 2556-02-31K | 484.6 | 190.8 | 174.7 | 187.0 | | | 7000 |
| 2556-02-31L | 287.7 | 173.7 | 161.7 | 160.6 | 155.1 | 154.4 | 8000 |
| 2556-02-31M | 486.5 | 182.9 | 172.1 | 171.7 | 168.3 | 163.8 | 7000 |

TABLE 6
Particle Distribution for KW-3902Emulsion (Lot 2556-02-31A)

| Number of Passes | Average size (nm) | Size Range (nm) |
|---|---|---|
| 0 | 259.1 | 98.8-525.0 |
| 1 | 132.8 | 63.1-237.6 |
| 2 | 126.8 | 62.1-222.8 |
| 3 | 126.2 | 67.2-210.1 |
| 4 | 130.1 | 72.9-204.9 |
| 5 | 113.9 | 53.4-205.5 |

TABLE 7
Particle Distribution for KW-3902Emulsion (Lot 2556-02-31B)

| Number of Passes | Average size (nm) | Size Range (nm) |
|---|---|---|
| 0 | 171.6 | 69.9-335.5 |
| 1 | 128.7 | 63.2-225.8 |
| 2 | 131.8 | 73.5-212.9 |
| 3 | 120.2 | 60.1-208.4 |
| 4 | 115.0 | 57.6-199.0 |
| 5 | 115.6 | 58.8-198.5 |

TABLE 8
Particle Distribution for KW-3902Emulsion (Lot 2556-02-31C)

| Number of Passes | Average size (nm) | Size Range (nm) |
|---|---|---|
| 0 | 197.0 | 83.4-377.1 |
| 1 | 136.2 | 69.0-234.3 |
| 2 | 127.8 | 66.6-215.9 |
| 3 | 126.8 | 67.0-212.2 |
| 4 | 121.3 | 62.3-206.8 |
| 5 | 120.5 | 65.3-198.5 |

TABLE 9
Particle Distribution for KW-3902Emulsion (Lot 2556-02-31D)

| Number of Passes | Average size (nm) | Size Range (nm) |
|---|---|---|
| 0 | 196.0 | 80.0-382.8 |
| 1 | 137.0 | 69.3-235.9 |
| 2 | 131.2 | 66.7-225.1 |
| 3 | 131.2 | 69.1-220.0 |
| 4 | 148.7 | 96.6-215.6 |
| 5 | 119.6 | 58.9-209.3 |

TABLE 10
Particle Distribution for KW-3902Emulsion (Lot 2556-02-31E)

| Number of Passes | Average size (nm) | Size Range (nm) |
|---|---|---|
| 0 | 255.0 | 117.0-465.8 |
| 1 | 123.1 | 62.5-211.2 |
| 2 | 115.5 | 27.8-200.0 |
| 3 | 115.2 | 59.2-196.4 |
| 4 | 107.3 | 53.4-186.5 |
| 5 | 105.5 | 53.0-182.5 |

TABLE 11
Particle Distribution for KW-3902Emulsion (Lot 2556-02-31F)

| Number of Passes | Average size (nm) | Size Range (nm) |
|---|---|---|
| 0 | 263.2 | 113.3-498.9 |
| 1 | 139.1 | 68.5-243.4 |
| 2 | 125.1 | 61.1-220.2 |
| 3 | 124.0 | 63.8-211.3 |
| 4 | 109.2 | 51.4-196.4 |
| 5 | 117.9 | 59.1-204.1 |

TABLE 12
Particle Distribution for KW-3902Emulsion (Lot 2556-02-31G)

| Number of Passes | Average size (nm) | Size Range (nm) |
|---|---|---|
| 0 | 442.2 | 171.6-887.7 |
| 1 | 170.2 | 151.2-190.7 |
| 2 | 172.8 | 130.4-223.1 |
| 3 | 165.8 | 108.0-240.0 |
| 4 | 157.0 | 139.4-175.9 |
| 5 | 152.2 | 135.2-170.5 |

TABLE 13
Particle Distribution for KW-3902Emulsion (Lot 2556-02-31H)

| Number of Passes | Average size (nm) | Size Range (nm) |
|---|---|---|
| 0 | 203.5 | 100.6-355.3 |
| 1 | 158.2 | 58.3-326.5 |
| 2 | 153.6 | 72.0-277.1 |
| 3 | 153.1 | 75.4-268.0 |
| 4 | 154.1 | 69.6-276.1 |
| 5 | 149.5 | 71.8-265.6 |

TABLE 14
Particle Distribution for KW-3902Emulsion (Lot 2556-02-31I)

| Number of Passes | Average size (nm) | Size Range (nm) |
|---|---|---|
| 0 | 349.4 | 162.8-632.5 |
| 1 | 177.4 | 84.0-317.9 |
| 2 | 161.0 | 78.8-282.7 |
| 3 | 159.0 | 77.3-280.6 |
| 4 | 154.8 | 89.2-244.5 |
| 5 | 149.1 | 93.1-222.7 |

TABLE 15
Particle Distribution for KW-3902Emulsion (Lot 2556-02-31J)

| Number of Passes | Average size (nm) | Size Range (nm) |
|---|---|---|
| 0 | 393.2 | 181.9-715.1 |
| 1 | 181.6 | 85.6-326.4 |
| 2 | 169.2 | 92.4-277.1 |
| 3 | 153.3 | 136.2-171.8 |

TABLE 16
Particle Distribution for KW-3902Emulsion (Lot 2556-02-31K)

| Number of Passes | Average size (nm) | Size Range (nm) |
|---|---|---|
| 0 | 484.6 | 190.1-967.5 |
| 1 | 190.8 | 97.7-325.9 |
| 3 | 174.7 | 89.0-299.2 |

TABLE 17
Particle Distribution for KW-3902Emulsion (Lot 2556-02-31L)

| Number of Passes | Average size (nm) | Size Range (nm) |
|---|---|---|
| 0 | 287.7 | 121.6-551.2 |
| 1 | 173.7 | 85.0-305.1 |
| 2 | 161.7 | 83.2-275.3 |
| 3 | 160.6 | 77.6-284.6 |
| 4 | 151.1 | 75.1-274.3 |
| 5 | 154.4 | 77.4-267.2 |

TABLE 18
Particle Distribution for KW-3902Emulsion (Lot 2556-02-31M)

| Number of Passes | Average size (nm) | Size Range (nm) |
|---|---|---|
| 0 | 486.5 | 209.0-923.6 |
| 1 | 182.9 | 84.5-332.8 |
| 2 | 172.1 | 86.8-296.9 |
| 3 | 171.7 | 82.2-305.7 |
| 4 | 168.3 | 87.4-285.0 |
| 5 | 163.8 | 81.2-285.6 |

TABLE 19
Comparison of Particle Size at Different Stages of Emulsification

| | Mean Diff. | | | 95% CI |
|---|---|---|---|---|
| Stage of | in Particle | | | of Diff. in |
| Emulsification | Size (nm) | t | P value | Part. Size |
| Premix v. 1 Pass | 150.6 | 7.41 | P < 0.001 | 88.68 to 212.4 |
| Premix v. 2 Pass | 159.0 | 7.82 | P < 0.001 | 97.09 to 220.9 |
| Premix v. 3 Pass | 161.1 | 7.93 | P < 0.001 | 99026 to 223.0 |
| Premix v. 4 Pass | 169.9 | 7.79 | P < 0.001 | 103.5 to 236.3 |
| Premix v. 5 Pass | 173.9 | 8.19 | P < 0.001 | 109.3 to 238.6 |
| 1 Pass v. 2 Pass | 8.4 | 0.41 | P > 0.05 | −53.47 to 70.30 |
| 1 Pass v. 3 Pass | 10.6 | 0.52 | P > 0.05 | −51.30 to 72.47 |
| 1 Pass v. 4 Pass | 19.3 | 0.89 | P > 0.05 | −47.03 to 85.70 |
| 1 Pass v. 5 Pass | 23.4 | 1.10 | P > 0.05 | −41.26 to 88.01 |
| 2 Pass v. 3 Pass | 2.2 | 0.11 | P > 0.05 | −59.71 to 64.05 |
| 2 Pass v. 4 Pass | 10.9 | 0.50 | P > 0.05 | −55.44 to 77.28 |
| 2 Pass v. 5 Pass | 15.0 | 0.70 | P > 0.05 | −49.67 to 79.59 |
| 3 Pass v. 4 Pass | 8.8 | 0.40 | P > 0.05 | −57.61 to 75.11 |
| 3 Pass v. 5 Pass | 12.8 | 0.60 | P > 0.05 | −51.84 to 77.42 |
| 4 Pass v. 5 Pass | 4.0 | 0.18 | P > 0.05 | −64.89 to 72.98 |

The initial passage of the emulsion through a microfluidizer had a large effect an average particle size. Subsequent passes through a microfluidizer did not significantly affect the average particle size. However, increasing the number of passes through the microfluidizer decreased the distribution of the particle size.

We claim:

1. A method of producing an emulsion for intravenous injection of a water-insoluble pharmaceutical composition, comprising,
mixing an oil, a first surfactant, a stabilizer, and said water-insoluble pharmaceutical composition to obtain a first mixture;
homogenizing said first mixture in a high shear homogenizer having a rotation speed to produce an emulsion having a first average particle size, wherein said homogenizing takes place in a bath at a temperature;
adjusting the pH of said emulsion to a target pH by addition of base or acid to said emulsion; and
determining a final pH of said emulsion;
wherein said target pH, said rotation speed, and said bath temperature are adjusted such that said final pH is between 5 and 7.

2. The method of claim 1, further comprising,
reducing the average particle size of said emulsion from said first average particle size to a second average particle size by passing said emulsion through a microfluidizer at least once, thereby obtaining a final emulsion.

3. The method of claim 1, wherein said water insoluble pharmaceutical composition comprises an adenosine A₁ receptor antagonist.

4. The method of claim 3, wherein said adenosine A₁ receptor antagonist is a xanthine derivative.

5. The method of claim 4, wherein said xanthine derivative is KW-3902.

6. The method of claim 1, wherein said oil is a natural triglyceride.

7. The method of claim 1, wherein said oil is a synthetic triglyceride.

8. The method of claim 7, wherein said synthetic triglyceride comprises at least one fatty acid chain greater than 8 carbons in length.

9. The method of claim 7, wherein said synthetic triglyceride comprises at least one fatty acid chain that is less than 22 carbons in length.

10. The method of claim 7, wherein said synthetic triglyceride comprises fatty acids with carbon chains of about 8-22 carbons in length.

11. The method of claim 6, wherein said natural triglyceride is a vegetable oil.

12. The method of claim 11, wherein said vegetable oil is soybean oil.

13. The method of claim 1, wherein said first surfactant is a phosphorus containing surfactant.

14. The method of claim 13, wherein said phosphorus containing surfactant is a naturally occurring phospholipid.

15. The method of claim 13, wherein said phosphorous-containing surfactant is phosphotidylcholine.

16. The method of claim 15, wherein said surfactant is egg yolk lecithin.

17. The method of claim 13, wherein said phosphorus containing surfactant is a PEG-phospholipid.

18. The method of claim 1, wherein said first surfactant is a block copolymer.

19. The method of claim 18, wherein said block copolymer comprises polyoxyethylene-polyoxypropylene.

20. The method of claim 1, wherein said stabilizer is an oncotic agent.

21. The method of claim 20, wherein said stabilizer is an oncotic agent selected from the group consisting of glycerin, saccharides, sugar alcohols, proteins and polypepties less than about 10 residues.

22. The method of claim 1, wherein said stabilizer comprises a nonionic surfactant.

23. The method of claim 22, wherein said nonionic surfactant is selected from the group consisting of a chelating agent, an antioxidant, a salt-forming counterion, a buffer, a sorbitan esters of a fatty acid, a polyethylene glycol ether, a polyetheleneglycol-sorbitan fatty acid ester, a fatty alcohol, and cholesterol.

24. The method of claim 1, further comprising a second surfactant.

25. The method of claim 24, wherein said second surfactant is an emulsifier.

26. The method of claim 25, wherein said emulsifier is an organic acid.

27. The method of claim 26, wherein said organic acid comprises greater than five carbon atoms.

28. The method of claim 26, wherein said organic acid comprises greater than ten carbon atoms.

29. The method of claim 26, wherein said organic acid comprises greater than fifteen carbon atoms.

30. The method of claim 26, wherein said organic acid comprises at least one double bond.

31. The method of claim 26, wherein said organic acid is oleic acid.

32. The method of claim 25, wherein said emulsifier is a mono-glyceride.

33. The method of claim 32, wherein said mono-glyceride is acetylated.

34. The method of claim 25, wherein said emulsifier is a di-glyceride.

35. The method of claim 25, wherein said emulsifier comprises a mixture of a polyethyleneglycol-sorbitan fatty acid ester and a sorbitan fatty acid ester.

36. The method of claim 1, wherein said target pH is at least 6.0.

37. The method of claim 1, wherein said target pH is at least 6.3.

38. The method of claim 1, wherein said target pH is at least 7.0.

39. The method of claim 1, wherein said target pH is at least 7.3.

40. The method of claim 1, wherein said target pH is at least 7.5.

41. The method of claim 1, wherein said target pH is at least 8.0.

42. The method of claim 1, wherein said target pH is at least 8.5.

43. The method of claim 1, wherein said target pH is at least 9.0.

44. The method of claim 1, wherein said rotation speed is between 5000 and 18,000 rotations per minute (rpm).

45. The method of claim 1, wherein said rotation speed is between 6000 and 9000 rpm.

46. The method of claim 1, wherein said rotation speed is between 7000 and 8000 rpm.

47. The method of claim 1, wherein said bath temperature is at least 25° C.

48. The method of claim 1, wherein said bath temperature is at least 30° C.

49. The method of claim 1, wherein said bath temperature is at least 35° C.

50. The method of claim 1, wherein said bath temperature is at least 40° C.

51. The method of claim 1, wherein said bath temperature is no more than 45° C.

52. The method of claim 2, wherein said crude emulsion is passed through a microfluidizer at least five times.

53. The method of claim 2, wherein said crude emulsion is passed through a microfluidizer at least three times.

54. The method of claim 2, wherein said crude emulsion is passed through a microfluidizer at least two times.

55. The method of claim 1, wherein said first average particle size is at least 100 nm.

56. The method of claim 1, wherein said first average particle size is at least 150 nm.

57. The method of claim 1, wherein said first average particle size is at least 200 nm.

58. The method of claim 1, wherein said first average particle size is at least 250 nm.

59. The method of claim 1, wherein said first average particle size is at least 300 nm.

60. The method of claim 1, wherein said first average particle size is at least 350 nm.

61. The method of claim 1, wherein said first average particle size is at least 400 nm.

62. The method of claim 1, wherein said first average particle size is at least 450 nm.

63. The method of claim 2, wherein said second average particle size is at least 100 nm.

64. The method of claim 2, wherein said second average particle size is at least 110 nm.

65. The method of claim 2, wherein said second average particle size is at least 120 nm.

66. The method of claim 2, wherein said second average particle size is at least 130 nm.

67. The method of claim 2, wherein said second average particle size is at least 140 nm.

68. The method of claim 2, wherein said second average particle size is at least 150 nm.

69. The method of claim 2, wherein said second average particle size is at least 160 nm.

70. A method of producing an emulsion for intravenous injection of a water-insoluble pharmaceutical composition, comprising,
mixing an oil, a first surfactant, a stabilizer, and said water-insoluble pharmaceutical composition to obtain a mixture;
adjusting the pH of said mixture to a first pH by addition of base or acid to said mixture;
homogenizing said mixture in a high shear homogenizer having a rotation speed to produce an emulsion having a first average particle size, wherein said homogenizing takes place in a bath at a temperature; and
determining a final pH of said emulsion;
wherein said first pH, said rotation speed, and said bath temperature are adjusted such that said final pH is between 5 and 7.
